(19) United States
(12) Patent Application Publication
(10) Pub. No.: US 2006/0252998 A1
(43) Pub. Date: Nov. 9, 2006

(54) MOBILE PHONE EMERGENCY MEDICAL DATA APPLICATION

(75) Inventor: Jacob Kimbrell, Raleigh, NC (US)

Correspondence Address: MOORE AND VAN ALLEN PLLC FOR SEMC, P.O. BOX 13706, 430 DAVIS DRIVE, SUITE 500, RESEARCH TRIANGLE PARK, NC, 27709, US

(73) Assignee: SONY ERICSSON MOBILE COMMUNICATIONS AB, Lund (SE)

(21) Appl. No.: 10/908,243

(22) Filed: May 4, 2005

(51) Int. Cl. A61B 5/00

(52) U.S. Cl. 600/300

(57) ABSTRACT

A mobile phone that can store and display personal medical information is presented. The mobile phone includes a medical data software application executable by a digital processor within the mobile phone. A storage device containing the personal medical information is coupled with the digital processor and a user interface is used to access and manipulate the medical data software application and personal medical information. The medical data software application is also coupled with a communications module to allow remote wireless access to the medical data application from another device.

### BACKGROUND

Currently, the only emergency feature a mobile station (e.g., cellular phone) provides is the ability to dial 911. In some mobile stations, a global positioning system (GPS) module may be included to assist in locating the mobile station and its user. In the event of an emergency, especially a medical emergency, it is often useful if those in a position to assist have access to particular medical history information about the injured or sick person. For example, emergency personnel will often search a persons wallet or purse seeking medical information that might be helpful in rendering aid. Things searched for include current medications, allergic conditions, or medical conditions such as diabetes, a pacemaker, etc. A user's mobile station is capable of storing much of this information in a way that can be accessed when necessary. This can lead to improved communication between the injured or sick party and the emergency personnel attempting to assist.

What is needed is a way to store and present relevant medical information pertaining to the user on a mobile station in the event of an emergency.

### SUMMARY

The present invention allows a mobile phone to store and display personal medical information. To accomplish the task, the mobile phone includes a medical data software application executable by a digital processor. A storage device containing the personal medical information is coupled with the digital processor. A user interface is used to access and manipulate the medical data software application and personal medical information. The user interface includes means for inputting data into the mobile phone under the direction of the medical data software application as well as means to display the personal medical information on the mobile phone's display.

The medical data software application is also coupled with a communications module to allow remote wireless access to the medical data application from another device. The other device can be used as a data input mechanism or, as a destination for personal medical information that is sent from the mobile phone. Provisions for securing access to the personal medical information are included and prompt anyone attempting to access the medical data software application to input an access code. External wireless devices that attempt to access the medical data software application are asked to authenticate themselves before any personal medical data can be exchanged.

### BRIEF DESCRIPTION OF THE DRAWINGS

FIG. 1 is an iconic view of the main menu selections for a mobile phone.

FIG. 2 is a view of an access verification screen used to grant access to personal medical data pertaining to the mobile phone owner (user).

FIG. 3 is a textual sub-menu pertaining to the medical icon selection of the main menu.

FIG. 4 is a detailed listing of the data for one of the sub-menu selections listed in FIG. 3.

FIG. 5 is a block diagram illustrating the communication between a user's mobile phone and an EMT module.

FIG. 6 is a flowchart illustrating the medical information configuration process.

FIG. 7 is a flowchart illustrating the medical information emergency access process when the mobile phone user is able to assist the EMT personnel.

FIG. 8 is a flowchart illustrating the medical information emergency access process when the mobile phone user is unable to assist the EMT personnel.

FIG. 9 is a block diagram illustrating the components within the mobile phone that carry out the present invention.

### DETAILED DESCRIPTION

The present invention is illustrated using a “soft” key access methodology integrated into a software menu function. Alternatively, the mobile station can recognize a pattern of key presses that automatically launch the medical data application. Other options for implementing a medical information key include an extra key that can be incorporated into the mobile station and designated as the medical information key. Most mobile stations also allow users to “program” a physical key to perform an action. The programming of a key acts as a shortcut eliminating the need for the user to navigate through a menu to find and activate the desired function. Thus, a user can program a shortcut key to present medical information. To further identify a “hot” or programmed key as the medical information key, the user can affix a small label or sticker to the key. The label could be a medical symbol like those seen on medical alert bracelets.

The terms user, owner, and victim are used throughout this disclosure and are intended to refer to the same person with respect to a mobile phone. The term victim is applied to describe situations where the owner/user of the mobile phone has suffered an injury or illness that requires emergency medical attention.

The medical information capable of being stored on the mobile phone includes, but is not limited to, emergency contact information, current medications and dosages, blood type, age, names of doctors, and medical history information. This information can be presented in a variety of ways. For instance, there can be a top level menu item for medical data. Under this item can be several sub-items for contact names, doctor names, medications, vital statistics (e.g., blood type), medical conditions (e.g., diabetes), and family medical history. Selecting any of these items will cause the mobile phone to display the data tied to that selection. If appropriate, further sub-menu items can provide further detail on a particular topic.

Personal medical data can be extremely sensitive and should be treated with great care. A mobile phone user would likely want to protect this information from inadvertent or intentional disclosure to third parties that are not in a position to assist in an emergency. The mobile phone user can protect the information by creating a unique access code. Successful entry of the access code can then be made a prerequisite to viewing the personal medical information contained on the mobile phone. This arrangement will work in most instances except when the user is unable to provide the access code due to unconsciousness or other disabling condition.

Thus, the medical information feature of the mobile phone can be incorporated as a module. The module can be RF (e.g., Bluetooth™) or infrared (IR) activated by emergency medical technician (EMT) personnel. EMT personnel can include paramedics, nurses, or doctors. The medical information module of the mobile phone would be accessible to EMT personnel that had an RF or IR device. The RF or IR device acts as a “key” to unlock the medical information contained in the mobile phone and allow the mobile phone to display the information.

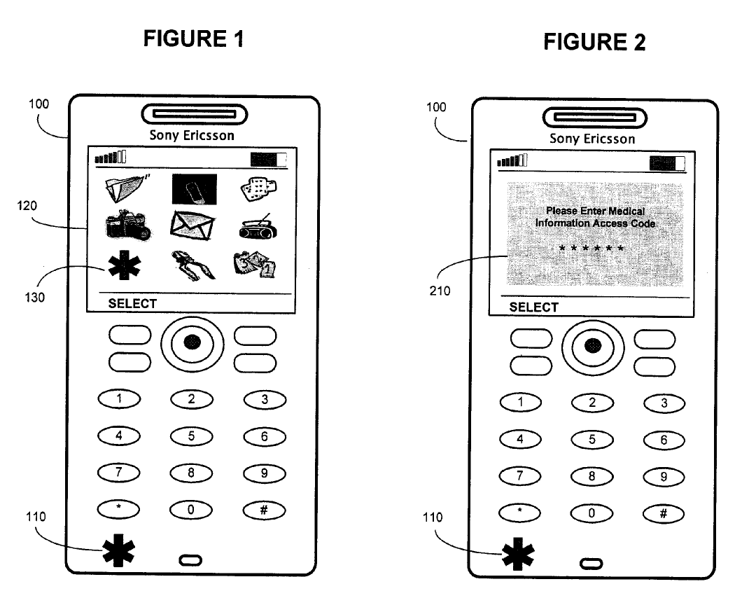

FIG. 1 is an iconic view of the main menu selections 120 for a mobile phone 100. Most mobile phones use a combination of icons and text menus that allow a user to navigate through the phone to find a desired application. FIG. 1 illustrates an example of a main or top-level menu displayed in iconic format. Each icon is representative of a function or application that has been programmed into the mobile phone 100. It should be noted that the icons shown in FIG. 1 are merely illustrative. The display of icons in FIG. 1 is not intended to be limiting to the present invention. One of ordinary skill in the art will readily understand that the number and type of icons can be altered without affecting the present invention.

Of interest to the present invention is the icon 130 in the lower left corner of the mobile phone display. This icon 130 is representative of the mobile phone's medical data feature. The same icon is also shown on the lower left corner 110 of the housing of the mobile phone 100. This can provide EMT personnel with a quick indication that the user/victim's mobile phone contains medical information that may assist in treatment.

FIG. 2 is a view of an access verification screen 210 used to grant access to personal medical data pertaining to the mobile phone owner (victim/user). This screen is accessed by selecting the medical icon 130 of FIG. 1. The access verification screen 210 prompts for a pre-determined access code to be entered. The mobile phone 100 will not grant further access to sensitive medical data unless this access code is successfully input. It is hoped that the user/victim will be able to either enter this access code on their own or provide the code to someone else who can physically enter it into the mobile phone. It is recognized that in some situations, the user/victim may not be able to provide this access code. There is a mechanism described later to address such a situation.

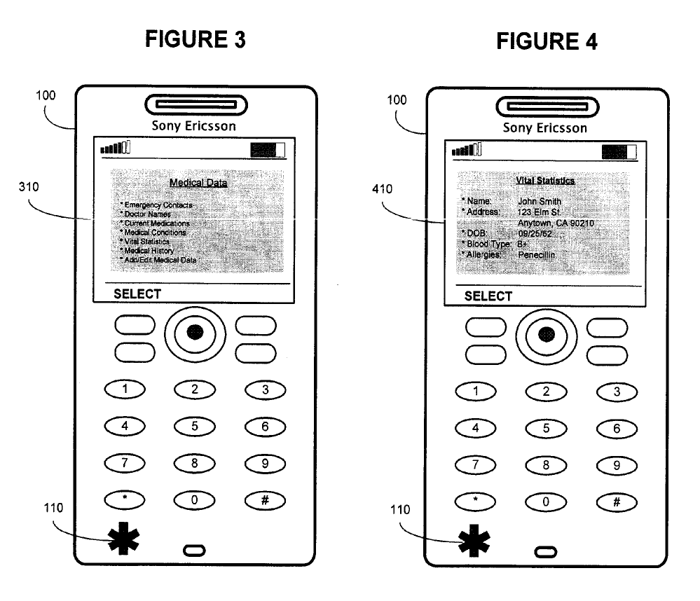

FIG. 3 is a textual sub-menu 310 pertaining to the medical icon selection of the main menu. This menu 310 is displayed upon successful entry of the access code described above. The sub-menu is a list of common medical data categories that can assist EMT personnel when dealing with the sick or injured victim/user. This list is not intended to be exhaustive and should not be construed as limiting the present invention. Moreover, the order and arrangement of the categories shown carries no significance. In this example there are six medical categories listed including emergency contacts, doctor names, current medications, medical conditions, vital statistics, and medical history. The victim/user will have pre-programmed data into these categories in a configuration process to be described later. In addition, there is a menu selection or category labeled “Add/Edit Medical Data” that allows the user to add more medical data or edit the existing medical data stored by the application.

For instance, the user can have input his spouse along with one or more telephone numbers as an emergency contact. Other individuals such as relatives, friends, co-workers can also be included. The emergency contact list is preferably ordered according to the person the victim/user would like notified first.

Under “Doctor Names”, the user can enter his primary physician and any specialists he is currently in the care of along with contact information for those doctors. The “Current Medications” selection can contain the names and dosages of medications the victim/user currently takes. The “Medical Conditions” selection can list any ailments such as diabetes, allergies, blood type, pacemaker, hemophilia, etc. The “Vital Statistics” selection can list the name, address, age, allergies, and blood type of the victim/user. The “Medical History” selection can include much of the information that a user would include in a typical medical questionnaire such as family history, allergies, etc. The user's blood type and allergies have been included in multiple categories. While this may seem redundant, it may help the EMT personnel because they do not have to check every menu selection to find certain critical data if that data can be found on multiple menu selections.

The type of data described under each category above is illustrative only. Additional data and additional categories can be programmed into the mobile phone without departing from the scope of the invention.

FIG. 4 is a detailed listing of the data for the “Vital Statistics” sub-menu selection 410 listed in FIG. 3. This figure illustrates that the victim/user is named John Smith, lives at 123 Elm St in Anytown, Calif., was born on Sep. 25, 1962, has a blood type of B+ and is allergic to penicillin.

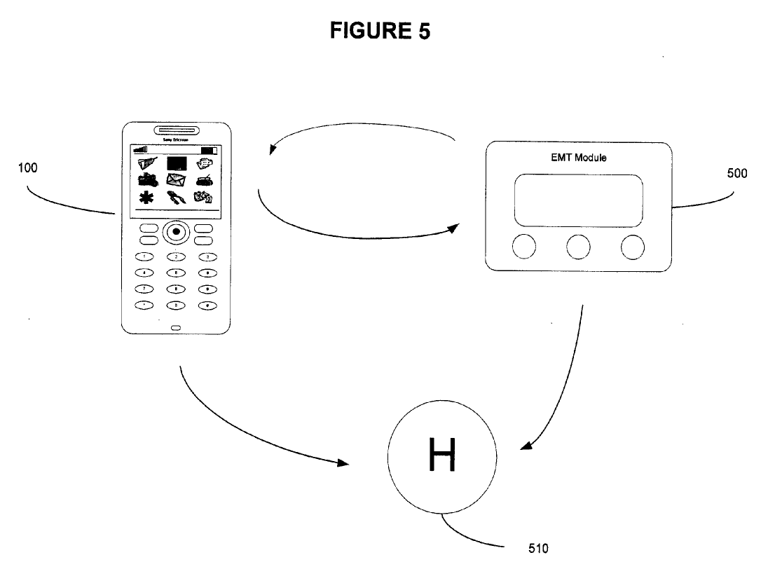

FIG. 5 is a block diagram illustrating the communication between a user's mobile phone and an EMT module. As mentioned earlier, sometimes obtaining the victim/user's assistance to access the medical data on the mobile phone may not be possible. In this situation, the access code can not be entered and the data can not be accessed. EMT personnel can still access this vital information if the mobile phone via an authorized module to module communication sequence. The entire medical data feature of the mobile phone 100 can be implemented as a module that supports a short range communication protocol such as RF Bluetooth or line of sight Infrared. The EMT personnel can carry a portable PDA like device equipped with a “master” module 500 that is communicable with the mobile phone's medical data module. The EMT device's module 500 can sync up with the mobile phone 100 and perform an authorization process to let the mobile phone know that the EMT device 500 is authorized to communicate with and download data from the mobile phone's medical data module. Since this can be performed without the victim/user's assistance, EMT personnel can still receive the critical medical information even if the victim/user is unconscious.

In addition, both the mobile phone and the EMT device module can further transfer the medical data to a hospital unit 510. This is especially useful if the victim/user requires further medical attention and must be transported to a hospital. The victim/user's medical data can be received and processed prior to the victim/user's arrival. This can save precious time when an attending emergency room doctor assesses a situation and decides on a course of action. The transfer of the medical data from the mobile phone 100 or the EMT device module 500 to a hospital unit 510 can be accomplished over a data network using techniques such as SMS messaging, MMS messaging, or any other wireless data transfer protocol that is supported by the sending and receiving devices.

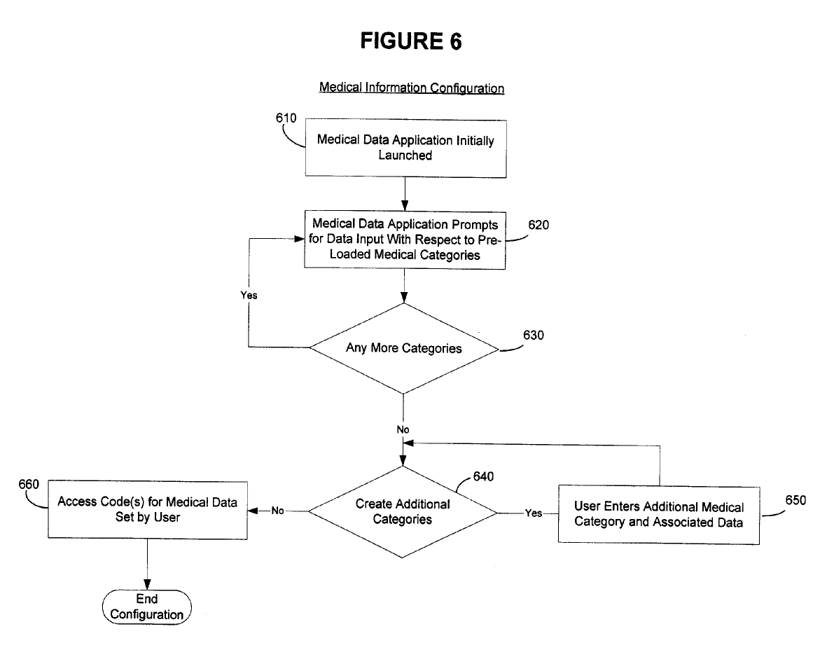

FIG. 6 is a flowchart illustrating the medical information configuration process. The medical information configuration process is essentially a data entry process by which the mobile phone user/owner inputs personal medical data into the medical information software application. This can be accomplished in a variety of ways. For example, upon initially launching the medical data application 610 the mobile phone user can be presented with a prompt asking the user to enter personal medical data 620. The medical information application can be pre-loaded with a plurality of medical categories like those described above. Each time the user enters data in response to a query, another query then appears 630 until all the pre-loaded medical categories have been populated with data. The user can also have the ability to add medical categories 640 that may not have been pre-loaded. If the user selects this option he defines a category and populates it with data 650.

Once all data entry has been completed, the last step of the configuration process is to set an access code 660. The medical data application will prompt the user to enter an access code that serves as a password to prevent unauthorized access to the personal medical data. Any future attempts to launch the medical data application will then require entry of the access code.

An alternative to configuring the medical data application directly on the mobile phone is to “port” the data from another device to the mobile phone. This may be desirable since data entry on a mobile phone can be a tedious cumbersome process. Software for configuring the medical application can be loaded on a computer such as a PC. This software can guide the user through he aforementioned configuration/data entry process using a full size keyboard and mouse device as data entry tools. This would greatly reduce the complexity and time needed to populate the data fields associated with the medical data application. Once the data has been entered on the computer, the computer can be coupled with the mobile phone via a cabled connection (e.g., USB, serial) or a wireless connection (e.g., RF, infrared) and the data can be downloaded into the mobile phone's medical data application.

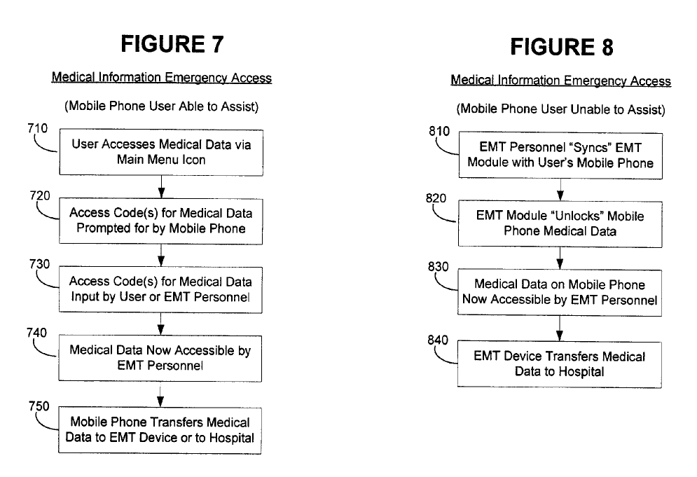

FIG. 7 is a flowchart illustrating the medical information emergency access process when the mobile phone user is able to assist the EMT personnel. If the mobile phone user falls victim to an accident or illness that requires emergency medical attention he can access the medical data on his mobile phone. First, the user will navigate the mobile phone's menu structure to find the medical data application icon. The user then launches the medical data application 710. The mobile phone then prompts for an access code 720. The user or EMT personnel then enters the access code 730 at the direction of the user. This unlocks the medical data application giving the EMT personnel full access to the medical data stored therein 740. During the course of treating the victim/user EMT personnel may decide that the victim/user need further attention at a hospital. In this case the EMT personnel and/or victim/user can have the mobile phone dump or download the medical data to the hospital so that the information can be assessed prior to the victim's arrival. This can save precious time in life threatening situations. The medical data can be dumped or downloaded to a designated hospital unit wirelessly using a data transfer standard such as, but not limited to, a series of short messaging service (SMS) text messages. The data can be collected at the hospital and re-formatted for hospital personnel. The data can also be forwarded to hospital administrative personnel to assist in filling out forms.

If the information is to be used by hospital administrative personnel, then another category can be included in the medical data application on the mobile phone that details the victim/user's health insurance data.

FIG. 8 is a flowchart illustrating the medical information emergency access process when the mobile phone user is unable to assist the EMT personnel. In this situation the EMT personnel would use a portable wireless device equipped with a medical data module. The module running on the EMT portable device would find and “sync up” with the victim's mobile phone medical data application module 810. The EMT device module would then unlock the medical data application in the mobile phone 820. The EMT device acts as a master key of sorts. Once unlocked the medical data on the mobile phone is accessible to the EMT personnel 830. The data can be viewed on the mobile phone or uploaded to the EMT device in a manner similar to that described above. Moreover, the EMT device can also forward the medical data it acquired from the mobile phone to a designated hospital unit just as above 840.

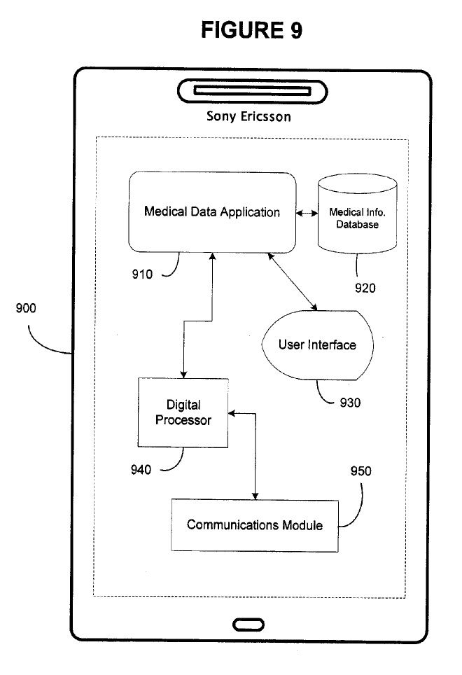

FIG. 9 is a block diagram illustrating the components within the mobile phone that carry out the present invention. The mobile phone 900 includes a medical data application 910 in the form of an executable software program. The medical data application 910 is linked with a database 920 or other memory storage device for storing the personal medical data of the user. The medical data application 910 is accessible to the user via a user interface 930 which is generically shown but typically includes both data entry mechanisms such as the mobile phone's keypad and data display mechanisms such as the mobile phone's display. The medical data application 910 is executed by a digital processor 940. A communications module 950 can also be incorporated to allow for wireless communication with other devices.

Computer program elements of the invention may be embodied in hardware and/or in software (including firmware, resident software, micro-code, etc.). The invention may take the form of a computer program product, which can be embodied by a computer-usable or computer-readable storage medium having computer-usable or computer-readable program instructions, “code” or a “computer program” embodied in the medium for use by or in connection with the instruction execution system. In the context of this document, a computer-usable or computer-readable medium may be any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium such as the Internet. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner. The computer program product and any software and hardware described herein form the various means for carrying out the functions of the invention in the example embodiments.

Specific embodiments of an invention are disclosed herein. One of ordinary skill in the art will readily recognize that the invention may have other applications in other environments. In fact, many embodiments and implementations are possible. The following claims are in no way intended to limit the scope of the present invention to the specific embodiments described above. In addition, any recitation of “means for” is intended to evoke a means-plus-function reading of an element and a claim, whereas, any elements that do not specifically use the recitation “means for”, are not intended to be read as means-plus-function elements, even if the claim otherwise includes the word “means”.

What is claimed is:

1. A mobile phone that can store and display personal medical information, the mobile phone comprising:
a digital processor;
a storage device containing the personal medical information coupled with the digital processor;
a medical data software application executable by the digital processor; and
a user interface for accessing and manipulating the medical data software application and personal medical information.

2. The mobile phone of claim 1 further comprising a communications module coupled with the digital processor, the communications module for providing remote wireless access to the medical data application from another device such that the personal medical information can be wirelessly sent to the other device.

3. The mobile phone of claim 2 wherein the medical data application authenticates the other device before allowing personal medical information to be wirelessly sent out from the mobile phone.

4. A medical data software application resident in a mobile phone and executable by the mobile phone and able to wirelessly exchange data with external devices, the medical data software application comprising:
a configuration component comprised of computer program code that collects and populates a database with personal medical information;
a secure access component comprised of computer program code that prevents unauthorized access to the personal medical information; and
a component comprised of computer program code that displays the personal medical information on the mobile phone display in response to user interface input.

5. The medical data software application of claim 4 further comprising a communications component comprised of computer program code that allows the personal medical information to be transmitted out of the mobile phone.
